(19) United States
(12) Patent Application Publication
(10) Pub. No.: US 2008/0119642 A1
(43) Pub. Date: May 22, 2008

(54) CONTROLLED SURFACE TOPOGRAPHY FOR ENHANCED PROTEIN CRYSTALLIZATION RATES

(75) Inventor: Matthew HALL, Alfred Station, NY (US)

Correspondence Address: BURR & BROWN, PO BOX 7068, SYRACUSE, NY, 13261-7068, US

(73) Assignee: ALFRED UNIVERSITY, Alfred, NY (US)

(21) Appl. No.: 11/943,134

(22) Filed: Nov. 20, 2007

Related U.S. Application Data
(60) Provisional application No. 60/866,697, filed on Nov. 21, 2006.

(51) Int. Cl. C07K 1/00

(52) U.S. Cl. 530/402

(57) ABSTRACT

A method for accelerating protein crystallization on a substrate is provided, including the steps of providing a coating layer comprising a colloidal solution containing inert particles on at least one discrete testing portion of a testing substrate to provide at least one coated portion, and drying the coated portion so that the coated portion has an enhanced surface topography defined by the characteristics of the coating layer. A supersaturated protein solution is applied to the coated portion, and the testing substrate is placed in an incubator for crystallization, and the growth rate of the protein crystals is accelerated during incubation due to the enhanced surface topography of the at least one coated portion. The testing substrate is evaluated to determine the degree of protein crystallization until crystallization in complete, and the protein crystals are subsequently removed from the testing substrate subjected to specific characterization testing.

### FIELD OF THE INVENTION

The present invention relates to methods for creating controlled surface topography to enhance protein crystallization and/or increase the rate of protein crystallization to facilitate the structural determination of the proteins by characterization methods such as X-ray diffraction, for example, so that the structural information may be applied to rational drug design.

### BACKGROUND OF THE INVENTION

Protein crystallization is a critical stage in the process of determining the structure of a protein by methods such as X-ray diffraction, for example. In order to obtain a protein crystal, the conditions of a protein solution are typically manipulated to achieve a state of super-saturation with respect to the protein, where the concentration of protein within the solution exceeds the solubility limit. Standard methods for encouraging the occurrence of protein crystallization include inducing changes in electrolyte concentration, pH, and the polarity of the solvent.

Current solutions to the problem include using porous materials, in bulk form, such as porous glass and porous silicon to provide a surface for protein crystal growth. There are, however, several drawbacks associated with using bulk porous glass immersion during protein crystallization. For example, the process of obtaining a protein crystal can be costly and inefficient, particularly in terms of the time required to screen many different solution conditions. In addition, it is difficult to integrate porous materials in bulk form with the current testing systems and equipment. In particular, optical transparency may be limited, which is not desirable, particularly with respect to the ability to perform the subsequent characterization testing.

The use of bioactive glass to accelerate protein crystallization has also been investigated. Such glasses are highly reactive when brought into contact with an aqueous solution which leads to the formation of a silica gel layer on the surface.

Using a reactive material, however, is not desirable, primarily because the dissolution products tend to alter the protein solubility. For example, the release of dissolution products into the protein-containing solution represents an uncontrolled variable that complicates and can hinder the process.

Another known method includes using specific minerals to enhance protein crystallization. For example, it has been found that, in some instances, proteins preferentially nucleate and grow from the surfaces of mineral particles. This enhancement of crystallization is attributed to an epitaxial process, whereby the dimensionality of the crystal structure of the mineral is geometrically compatible with the protein, such that the mineral serves as a “template” to guide the nucleation and growth of a protein crystal (see A. McPherson and P. Shlichta (1998). Heterogeneous and epitaxial nucleation of protein crystals on mineral surfaces. Science, vol. 239, pp. 385-387). A specific disadvantage associated with this method is that it is difficult to incorporate into a useful screening system.

A method for accelerating the protein crystallization process is highly desirable to improve the throughput and increase the number of different protein crystals that can be crystallized in discrete locations on a single plate for subsequent characterization. However, such a method has not heretofore been provided by the prior art.

### SUMMARY OF THE INVENTION

It is an object of the invention to provide enhanced and accelerated protein crystal growth to facilitate protein crystallization and the subsequent process of characterization of the protein crystals by methods such as X-ray diffraction. Such characterization information may then be used for the purpose of rational drug design, for example. The present invention can be easily incorporated into the existing technology and used as a higher-throughput, lower-cost alternative in connection with the devices that are currently used for conducting protein crystallization characterization.

The present invention provides significant advantages over the prior art in two important ways. First, the particulate coatings according to the present invention enhance and/or accelerate the crystallization of proteins. This significantly reduces the time required for crystallization and allows for faster results and an increased throughput. Secondly, the present invention can readily be integrated, without undue expense, into a format that is compatible with current methods employed by practitioners of protein crystallization.

The present invention provides a method for creating controlled surface topography to increase the rate of protein crystallization by providing a particulate coating on a substrate to enhance and/or accelerate protein crystallization.

The term “enhancement of protein crystallization” used herein refers to an increased tendency for crystallization to occur in the presence of the particulate coating, whereas the term “acceleration of protein crystallization” refers to a decrease in the time required for protein crystal nuclei to form.

According to a first aspect of the present invention, a method for accelerating protein crystallization on a substrate is provided, comprising the steps of providing a testing substrate having a testing surface with one or more discrete testing portions thereon, and providing at least one coating layer comprising a colloidal suspension containing chemically inert particles on at least one of the one or more testing portions to provide at least one coated portion. The method also includes a step of drying the at least one coating layer on the at least one coated portion so that the at least one coated portion has a surface topography that is defined by characteristics of the at least one coating layer and that differs from a surface topography of an uncoated portion of the testing substrate, providing a supersaturated protein solution and applying the supersaturated protein solution to the at least one coated portion of the testing substrate. Further, the method includes placing the testing substrate in an incubator and incubating the supersaturated protein solution to promote protein crystallization, wherein a growth rate of protein crystals grown during the incubating step is accelerated due enhancement of the surface topography of the at least one coated portion compared to the surface topography of an uncoated portion of the testing substrate, and periodically evaluating the testing substrate during the incubating step to determine a degree of protein crystallization until protein crystallization is complete.

According to a second aspect of the present invention, a method for accelerating protein crystallization on a substrate is provided comprising the steps of providing a testing substrate having a testing surface with a plurality of discrete testing portions thereon, providing at least one of a first coating layer comprising a first colloidal suspension containing chemically inert particles on one or more first discrete testing portions to form at least one first coated portion and providing at least one of a second coating layer comprising a second colloidal suspension containing chemically inert particles on one or more second discrete testing portions to form at least one second coated portion. The method also includes the steps of drying the at least one first and second coating layers so that the respective first and second coated portions have surface topography characteristics that differ from a surface topography of an uncoated portion of the testing substrate, providing a supersaturated protein solution, applying the supersaturated protein solution to the at least one first and second coated portions, and placing the testing substrate in an incubator and incubating the supersaturated protein solution to promote protein crystallization, wherein a growth rate of protein crystals grown during the incubating step is accelerated, due to the differing surface topography characteristics of the respective at least one first and second coated portions, compared to the surface topography of an uncoated portion of the testing surface. Further, the method involves periodically evaluating the testing substrate during the incubating step to determine a degree of protein crystallization until protein crystallization is complete to provide protein crystals and determining one or more characteristics of the protein crystals grown in the respective at least one first and second coated portions.

In the method according to the first aspect, the chemically inert particles in the colloidal suspension preferably have an average particle size of 10 μm or less, more preferably an average particle size of 1 μm or less.

In the method according to the second aspect, the chemically inert particles in each of the first and second colloidal suspensions preferably have an average particle size of 10 μm or less, and more preferably have an average particle size of 1 μm or less.

In the method according to the first and second aspects, the at least one coated portion of the first aspect and the at least one first and second coated portions of the second aspect each have an average pore size of 1 μm or less.

According to both aspects, it is preferred that the chemically inert particles in the respective colloidal suspensions each comprise a chemically stable material that is resistant to dissolution/corrosion in the protein solution.

According to one embodiment, the chemically inert particles in the respective colloidal suspensions of the first and second aspects each preferably comprise an oxide material comprising at least one of silica, zirconia, alumina and a complex oxide metal. It is also preferred that the oxide particles in the first colloidal suspension are different than the oxide particles in the second colloidal suspension in the method according to the second aspect.

It is also preferred that that each of the first and second aspects includes a step of rinsing the respective coated portions to remove impurities before the step of applying the protein solution.

According to a third aspect of the present invention a method for accelerating protein crystallization on a substrate is provided, comprising the steps of providing a testing substrate having a testing surface with a plurality of discrete testing portions thereon, providing at least one of a first coating layer on one or more first discrete testing portions to form at least one first coated portion and providing at least one of a second coating layer on one or more second discrete testing portions to form at least one second coated portion. The method also includes the steps of drying the at least one first and second coating layers so that the respective first and second coated portions have surface topography characteristics that differ from a surface topography of an uncoated portion of the testing substrate, providing a supersaturated protein solution, applying the supersaturated protein solution to the at least one first and second coated portions and placing the testing substrate in an incubator and incubating the supersaturated protein solution to promote protein crystallization, wherein a growth rate of protein crystals grown during the incubating step is accelerated due to enhanced surface topography characteristics of the at least one first and second coated portions compared to the surface topography of an uncoated portion of the testing surface. The method also includes the steps of periodically evaluating the testing substrate during the incubating step to determine a degree of protein crystallization until protein crystallization is complete to provide protein crystals and determining one or more characteristics of the protein crystals grown in the respective at least one first and second coated portions.

In the method according to the third aspect, at least one of the at least one first and second coating layers preferably comprises one of a porous oxide layer, a porous metal layer and a porous polymer layer. The porous oxide layer preferably comprises at least one of silica, zirconia, alumina and a complex oxide material, and it is preferred that the first and second coating layers are different from one another.

It should also be noted that the methods according to either or all of the first, second or third aspects also include providing at least one of a third coating layer on one or more third discrete testing portions to form at least one third coated portion that comprises a material that is different from a material of the respective first and second coating layers.

The functionality of the coating according to the present invention is due, at least in part, to the size of the porosity created by inter-particle voids of the particulate coating on the substrate, that is, the empty space that remains between particles that are packed together. The size of the porosity is controlled by the size of the particles used to form the coating. For example, as the average particle size increases, the average pore size also increases. Controlling the particle size and particle packing of the particles in the coating are the preferred methods for ensuring appropriate porosity. Particle packing may be influenced by adjusting characteristics of the particle suspension such as pH and solids loading, for example.

In order to achieve an appropriate porosity, or an appropriate pore size, the particles should be no larger than approximately 10 μm in diameter. Pores of the appropriate size enhance/accelerate protein crystallization by a combination of factors including interfacial curvature and spatial confinement of aggregated proteins. The appropriate pore size depends upon factors such as the size of the protein, for example, and would not ordinarily exceed a pore diameter of 1 μm as a practical upper limit.

It should be noted, however, that the texture of the coating begins to approximate a flat surface when the upper size limit of the pores of 10 μm is exceeded. Flat surfaces do not exhibit the same ability to accelerate protein crystallization as a textured coating, and crystallization rates are slower on flat surfaces without enhanced topography. The limit on the lower porosity size range is reached when the scale of the inter-particle voids becomes smaller than the protein. At this point, the surface roughness becomes too small, effectively rendering it “invisible” to proteins in solution.

It should be understood, however, that the actual pore size varies depending on the characteristics of the specific protein in question. In general, the average pore size should be at least as large as a single protein, and preferably one to two orders of magnitude larger than a single protein molecule. This specification is based on the premise that protein crystals grow from nuclei of a critical size to permit further growth. Such nuclei consist of multiple protein molecules that aggregate together to form an ordered arrangement. However, the critical nucleus size required to achieve crystal growth will vary from system to system.

The pore size of the coating layer influences the rate of crystallization and/or the tendency for the protein to crystallize. For example, the pore size affects the interfacial curvature, which, in turn influences the rate and/or tendency of a protein to crystallize by altering the conditions for solubility on a very local level. Interfacial curvature ties into a general set of behaviors known as “capillarity effects.” These general effects include the meniscus that is observed when a liquid is placed in a tube of sufficiently small diameter (such as a capillary). In the present invention, the conditions for protein solubility are considered within the vicinity of a roughened surface. The roughened surface consists of regions of positive curvature (e.g., hills) and regions of negative curvature (e.g., valleys). In general, protein solubility tends to decrease in regions of negative curvature (valleys). In the context of the present invention, the region of negative curvature is analogous to the “neck” that is formed when two particles come into contact. In the case of proteins within the vicinity of the porous coating, the pores act as regions of negative curvature that tend to reduce protein solubility.

The specific chemical identity of the particle used to form the coating also

influences the protein crystallization process. However, other particle chemistries and/or surface modifications can be used depending on the desired application, as discussed in more detail below.

The protein crystallization process is also dependent upon the protein solution in several important ways. First, the pH of the solution controls the electrical charging behavior of both the coating layer and protein. The isoelectric point/point of zero charge (PZC) of a material is a property that tends to influence the interaction between the coating layer and the protein in solution. For example, silica has a PZC of 2 to 3, which means that it will have a negative charge at pH values greater than 3. A positively charged protein might interact more strongly with a silica coating that is negatively charged. On the other hand, materials such as alumina or zirconia have PZC values in the range of 9 to 10.

A preferred embodiment includes a coating of colloidal silica particles having a diameter no greater than approximately 1 μm so as to promote the proper pore size for the desired surface topography. The benefits of silica include chemical durability, purity, optical transparency, and availability in numerous particle sizes. According to another aspect of the present invention, zirconia or alumina particles are used as the coating material, for instance, in cases where alkaline pH resistance is desired. Examples of other suitable materials include any oxide material with sufficient chemical durability in aqueous solutions having pH values between 4 to 10. That is, the particles can ideally be any material that is chemically stable (i.e., resistant to dissolution/corrosion) in the protein solution used for the protein crystallization experiment. While the preferred embodiment described herein involves the use of a chemically stable oxide material such as silica, alumina, titania, zirconia, a very thin porous film of a metal or plastic could also be deposited to enhance the surface topography of the growth surface, provided that such a metal or plastic coating does not interfere with the optical transparency of the multi-well plate.

In addition, complex oxides such as mullite (e.g., an aluminosilicate) could also be used as a coating. In this case, the ability to use multi-component oxides provides an advantage by being able to achieve coating properties that lie somewhere between the properties exhibited by pure silica and pure alumina. For example, the surface charge of an oxide material can be regarded as a blend or average of the individual, constituent oxides, e.g., silica tends to be negatively charged at a neutral pH while alumina is positively charged. By producing an aluminosilicate compound, the surface charge at neutral pH will typically be intermediate between the two extremes.

With respect to usage, an intermediate compound would be used in the same manner as pure oxides, so long as sufficiently small colloidal particles of the appropriate composition can be provided. These colloidal particles of the intermediate compound are then deposited to form a coating on the multi-well plate in the same manner described in connection with the preferred embodiment discussed below.

Different types of coating particles, each having distinct chemistry and/or size characteristics, may be applied and used on discrete portions of the same substrate or multi-well plate to provide a plurality of differing protein crystallization conditions. In that manner, a number of protein crystals can be grown on a single testing medium, like a micro-titer plate, using different types of coating particles for to provide different growth conditions. Using multiple coating types on a single device increases and enhances the capacity for crystallizing a more diverse range of proteins. Preferably, the different coating particle materials should have distinct PZC properties.

The material of the substrate upon which the particulate coating is applied is not limited, although a preferred embodiment of the present invention involves the use of a polymer substrate typically used in the manufacturing of standard laboratory ware (e.g., polystyrene). Other suitable examples include, but are not limited to glass, ceramic, or metallic substrates. In particular, it is preferable that the substrate exhibits hydrophilic characteristics to facilitate the application of the coating thereon.

The overall structure of the substrate is also not limited, however, a preferred embodiment of the present invention includes a substrate having the form of a multi-well plate. For example, the particulate coating may be applied to the bottom inner surfaces of the wells within a micro-titer plate, or to a predetermined portion the testing surface of another item of general laboratory ware such as a cover-slip, side mount or petri dish.

While the coating thickness does not significantly impact the ability of the coating to influence protein crystallization, coating thickness does impact at least two other parameters, those being optical clarity and coating stability (particularly during drying). For example, since the coating layer is porous, a thicker coating eventually obtains a hazy appearance. This is not desirable, however, because an optically transparent coating is preferred because optical microscopy is used to monitor for the presence of protein crystals.

The thickness of the coating also influences the successful drying of the porous coating. As coating thickness increases, the stress that develops within the coating also increases. If the stress within the coating becomes too high, the coating will crack and/or delaminate from the surface. As a general rule, a coating can be up to about 1 μm in thickness without incurring damage during the drying process. Of course, it is also possible to use a multi-step process—that is, apply one coating, let it dry, apply a second coating on top of the first, let it dry, etc., to obtain a thicker coating, if such is desired.

The particle coating is preferably a liquid suspension containing the particles for the coating that is applied onto the substrate with a pipette or similar delivery device. The liquid is removed from the particulate suspension by an appropriate drying process, as discussed above. One or more washes may be subsequently used to remove soluble impurities, followed by a final drying step.

Other methods of coating the colloidal particles include, but are not limited to spraying the suspension onto the substrate and applying dry particles through an electrostatic process or by the prior application of an adhesive.

It is critical to prevent uncontrolled evaporation from the protein solution, since changes in protein concentration will directly impact solubility and affect the subsequent crystallization. Pressure and moisture/humidity do not pose a threat provided that the system is sealed reasonably well. For instance, methods for sealing a 96 well plate are known.

### BRIEF DESCRIPTION OF THE DRAWINGS

FIG. 1 is a schematic diagram of a preferred embodiment of the invention, showing a plurality of different types of colloidal particles deposited onto the

bottom inner surfaces wells within a micro-titer plate for protein crystallization thereon.

FIG. 2 is a cross-sectional view taken through like II-II in FIG. 1 showing the coating in the wells.

FIG. 3 is a schematic diagram of another embodiment of the present invention showing a coating of colloidal particles provided on a predetermined portion of a cover slip for protein crystallization thereon.

### DETAILED DESCRIPTION OF THE INVENTION

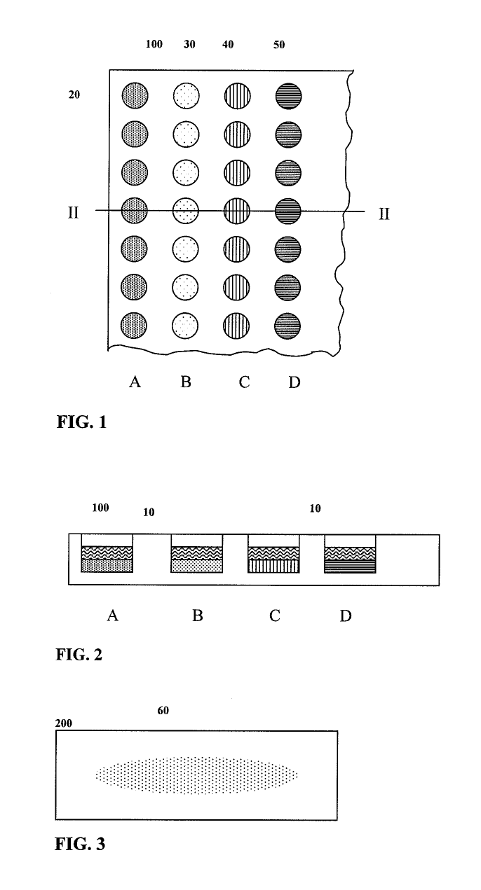

FIG. 1 is a partial schematic diagram of a preferred embodiment of the invention, showing a plurality of different types if colloidal particles deposited onto the bottom inner surface of a well within a micro-titer plate for protein crystallization thereon.

A plate 100 containing a plurality of wells, for example, 96 wells, is provided, and each column (or row) is coated by a unique coating material. For example, the wells 20 in column A are provided with a silica coating, whereas the wells 30 in column B are coated with zirconia. The column or rows can be coated so that alternating ones have different coatings or repeating patterns of the same coating materials.

As shown, the wells 40 and 50 in columns C and D are also coated with different coatings, such as alumina or other suitable coating materials described above. Using several different types of coatings on a single plate as shown here provides a format that allows for higher throughput screening of possible protein crystallization conditions.

FIG. 2 is a cross-sectional view taken through line II-II in FIG. 1 showing the coated portions in the wells of each column as well as the protein solution 10 deposited thereon. As shown, the same protein solution 10 is applied to each of the columns in the multi-well plate. In screening, the same protein solution could be applied to each of the different types of coating materials, and the presence or absence of crystals would then be noted for each coating type.

FIG. 3 is a schematic diagram of another embodiment of the present invention showing a coating of colloidal particles 60 provided on a predetermined portion of a cover slip 200 for protein crystallization thereon.

The preferred method according to the present invention includes the steps of depositing a suspension of colloidal particles, such as silica particles, for example, directly into the wells of a multi-well plate. The liquid is then removed by drying. In practice, the drying temp is dictated by considering the heat compatibility of substrate. For example, some coatings can be sufficiently dried at a temperature of less than 100° C. over a period of several hours. A subsequent series of rinsing steps is used to remove residual contaminants (e.g., sodium), thereby leaving a nominally pure silica coating. The rinse may consist of an acidic solution to help solubilize potential contaminants, followed by one or more rinses with deionized water.

A supersaturated solution containing dissolved protein of a known concentration is placed on the coating within the wells of the multi-well plate. The solution also contains various salts, buffering compounds, etc. Suitable examples of such salts and buffering compounds include, but are not limited to, and often depend on the particular characteristics of the protein being crystallized. This assembly is then placed within an incubator that maintains a constant temperature. The assembly is periodically removed from the incubator to check for the presence of crystals using optical microscopy. Again, the incubating time and temperature depend on the individual protein in question and the thermal resistance characteristics of the substrate material.

The crystallization rate relates to the time required to grow a complete crystal. For example, a protein crystal of about 100 μm would be considered to be complete. The present invention improves the rate at which the proteins reach a state of completed crystallization. In addition, the quantity of crystals could also be enhanced, although a large number of crystals are not necessarily desirable. The time required to crystallize a protein can vary from hours to weeks, depending upon experimental conditions. In that manner, the crystallization rate is accelerated in relative terms.

The multi-well plate is periodically removed from the incubator, viewed under an optical microscope, and evaluated to determine whether a sufficient crystal size is present to indicate that crystallization is complete. The high throughput nature of screening step relates to the ability to significantly increase the number of differing solution conditions that can be simultaneously tested. The subsequent XRD characterization, however, still proceeds one sample at a time, after the individual crystals are removed from their respective portions of the testing substrate.

While the present invention has been explained herein by way of example, its should be understood that scope of the present invention is in no way limited to these examples, and can be modified without departing from the spirit of invention.

We claim:

1. A method for accelerating protein crystallization on a substrate comprising the steps of:
providing a testing substrate having a testing surface with one or more discrete testing portions thereon;
providing at least one coating layer comprising a colloidal suspension containing chemically inert particles on at least one of the one or more testing portions to provide at least one coated portion;
drying the at least one coated portion so that the at least one coated portion has a surface topography that is defined by characteristics of the at least one coating layer and that differs from a surface topography of an uncoated portion of the testing substrate;
providing a supersaturated protein solution;
applying the supersaturated protein solution to the at least one coated portion of the testing substrate;
placing the testing substrate in an incubator and incubating the supersaturated protein solution to promote protein crystallization, wherein a growth rate of protein crystals grown during the incubating step is accelerated due to an enhancement of the surface topography of the at least one coated portion compared to the surface topography of an uncoated portion of the testing substrate; and
periodically evaluating the testing substrate during the incubating step to determine a degree of protein crystallization until protein crystallization is complete.

2. A method for accelerating protein crystallization on a substrate comprising the steps of:
providing a testing substrate having a testing surface with a plurality of discrete testing portions thereon;
providing at least a first coating layer comprising a first colloidal suspension containing chemically inert particles on one or more first discrete testing portions to form at least one first coated portion;
providing at least a second coating layer comprising a second colloidal suspension containing chemically inert particles on one or more second discrete testing portions to form at least one second coated portion;
drying the first and second coated portions so that the respective first and second coated portions each have surface topography characteristics that differ from a surface topography characteristic of an uncoated portion of the testing substrate;
providing a supersaturated protein solution;
applying the supersaturated protein solution to the first and second coated portions;
placing the testing substrate in an incubator and incubating the supersaturated protein solution to promote protein crystallization, wherein a growth rate of protein crystals grown during the incubating step is accelerated due to the differing surface topography characteristics of the respective first and second coated portions, compared to the surface topography characteristics of an uncoated portion of the testing surface;
periodically evaluating the testing substrate during the incubating step to determine a degree of protein crystallization until protein crystallization is complete to provide protein crystals; and
determining one or more characteristics of the protein crystals grown in the respective at least one first and second coated portions.

3. The method according to claim 1, wherein the chemically inert particles in the colloidal suspension have an average particle size of 10 μm or less.

4. The method according to claim 3, wherein the chemically inert particles in the colloidal suspension have an average particle size of 1 μm or less.

5. The method according to claim 2, wherein the chemically inert particles in each of the first and second colloidal suspensions have an average particle size of 10 μm or less.

6. The method according to claim 5, wherein the chemically inert particles in each of the first and second colloidal suspensions have an average particle size of 1 μm or less.

7. The method according to claim 1, wherein the at least one coated portion has an average pore size of 1 μm or less.

8. The method according to claim 2, wherein the at least one first and second coated portions each have an average pore size of 1 μm or less.

9. The method according to claim 1, wherein the chemically inert particles in the colloidal suspension comprise a chemically stable material that is resistant to dissolution/corrosion in the protein solution.

10. The method according to claim 2, wherein the chemically inert particles in each of the first and second colloidal suspensions comprise a chemically stable material that is resistant to dissolution/corrosion in the protein solution.

11. The method according to claim 9, wherein the chemically inert particles in the colloidal suspension comprise at least one oxide material selected from the group consisting of silica, zirconia, alumina and a complex oxide material.

12. The method according to claim 10, wherein the chemically inert particles in each of the first and second colloidal suspensions comprise at least one oxide material selected from the group consisting of silica, zirconia, alumina and a complex oxide material.

13. The method according to claim 12, wherein the oxide particles in the first colloidal suspension are different than the oxide particles in the second colloidal suspension.

14. The method according to claim 1, further comprising a step of rinsing the at least one coated portion to remove impurities before the step of applying the protein solution.

15. The method according to claim 2, further comprising a step of rinsing the at least one first and second coated portions to remove impurities before the step of applying the protein solution.

16. A method for accelerating protein crystallization on a substrate comprising the steps of:
providing a testing substrate having a testing surface with a plurality of discrete testing portions thereon;
providing at least one first coating layer on one or more first discrete testing portions to form at least a first coated portion;
providing at least a second coating layer on one or more second discrete testing portions to form at least one second coated portion;
drying the at least one first and second coated portions so that the respective first and second coated portions have surface topography characteristics that differ from a surface topography of an uncoated portion of the testing substrate;
providing a supersaturated protein solution;
applying the supersaturated protein solution to the first and second coated portions;
placing the testing substrate in an incubator and incubating the supersaturated protein solution to promote protein crystallization, wherein a growth rate of protein crystals grown during the incubating step is accelerated due to enhanced surface topography characteristics of the first and second coated portions compared to the surface topography characteristics of an uncoated portion of the testing surface;
periodically evaluating the testing substrate during the incubating step to determine a degree of protein crystallization until protein crystallization is complete to provide protein crystals; and
determining one or more characteristics of the protein crystals grown in the respective at least one first and second coated portions.

17. The method according to claim 16, wherein at least one first and second coating layers comprises one of a porous oxide layer, a porous metal layer and a porous polymer layer.

18. The method according to claim 17, wherein the porous oxide layer comprises at least one material selected from the group consisting of silica, zirconia, alumina and a complex oxide material.

19. The method according to claim 16, wherein respective compositions of the first and second coating layers are different from one another.

20. The method according to claim 16, further comprising providing at least a third coating layer on one or more third discrete testing portions to form at least one third coated portion, the third coating layer having a composition that is different from respective compositions of the first and second coating layers.
